(19) United States
(12) Patent Application Publication
(10) Pub. No.: US 2009/0221929 A1
(43) Pub. Date: Sep. 3, 2009

(54) Method and Arrangement for the Analysis of a Time-Variable Bioelectromagnetic Signal

(76) Inventors: Werner Alfona Scherbaum, Düsseldorf (DE); Jose Alberto Gonzales-Hernandez, Ciudad Habana (CU)

Correspondence Address: GUDRUN E. HUCKETT DRAUDT, SCHUBERTSTR. 15A, WUPPERTAL, 42289, DE

(21) Appl. No.: 11/997,604

(22) PCT Filed: Aug. 4, 2006
(86) PCT No.: PCT/DE06/01370

§ 371 Date: Sep. 22, 2008

(30) Foreign Application Priority Data
Aug. 4, 2005 (DE) 10 2005 037 326.7

(51) Int. Cl. A61B 5/0484; A61B 5/0488

(52) U.S. Cl. 600/544; 600/546

(57) ABSTRACT

In a method for analysis of a time-variable bioelectromagnetic signal that has been recorded over a certain time interval, the signal is split by a bandpass filter into at least two signal components that differ with regard to their frequency ranges. At least one reference point of a first kind is determined in at least one of the signal components that differ with regard to their frequency ranges in accordance with predetermined selection criteria. The values of at least two signal components that differ with regard to their frequency ranges at the determined reference points of the first kind are correlated with one another according to predetermined evaluation criteria.

### BACKGROUND OF THE INVENTION

The invention concerns a method and an arrangement for analysis of a bioelectromagnetic signal that changes over time. In particular, the invention concerns a method and an arrangement for analysis of electroencephalographic signals. In further independent claims, the invention concerns advantageously configured electroencephalographs, electromyographs, magnetoencephalographs and electroneurographs.

In this context, “bioelectromagnetic signals” are to be understood as those electric and/or magnetic signals that are detected by appropriate sensors and detectors, for example, electrodes, which signals originate from electrical activity of a biological object, for example, a beating heart or another muscle, the brain, or peripheral nerves. Since moving electrical charges induce magnetic fields, in the following the term electromagnetic signals is always used even though in many applications actually only an electrical potential or its change over time is measured.

### PRIOR ART

The detection and evaluation of bioelectromagnetic signals have greatly gained importance over the past years and have been employed in different fields, not only in research and medicine, but e.g. in regard to control of machines by humans without the use of muscle power. Other examples are the creation of intelligent prostheses that react to electromagnetic pulses originating in the brain of the user and carry out certain actions.

DE 43 27 429 A1 discloses a method for brain wave analysis in which the signals detected at the head of a human are divided by means of an analog or digital bandpass filter into signal components of different frequency ranges. It has been actually found that in the brain electrical pulses of frequencies that significantly differ from one another are exchanged between the neurons wherein the different frequency ranges usually are referred to as α, β, γ, and Θ ranges. Generally known is, for example, the so-called α state when the main brain activity takes place at frequencies in the α range and the human is in a relaxed state in which the human is particularly receptive to learning.

DE 693 30 644 T2 discloses a method for separating signal components of a time-variable multi-channel measured signal wherein the measured signals, for example, are evoked electrical and magnetic response signals, spontaneous activity signals of the brain, or measured signals received from the heart.

DE 198 19 497 A1 discloses a device for identification of heart and brain states based on different frequency spectral structures of the electromagnetic activities of the neurons enervating these organs, wherein the electromagnetic activities are repeatedly detected and supplied to an electrical device that transfers the received electromagnetic signals from a time spectrum to a frequency level.

DD 267 335 A1 discloses a switching arrangement for the analysis of an electroencephalogram with which the precision of electroencephalograms and thus of their diagnostic value is to be increased.

DD 299 509 A7 discloses a method for event-related non-linear topological functional analysis that is able to determine dynamic non-linear function-relevant topological parameters.

DE 692 28 823 T2 discloses a method for non-invasive detection of cerebral phenomena in which, after bandpass filtration of electroencephalographic signals, dynamic phase relations are characterized within the filtered signal.

In addition, scientific literature (for example, Duffy, F. H.: Topographic Mapping of Brain Electric Activity, Boston, Butterworth 1986, 7-28) discloses different methods for resolution of pulse responses to external stimuli and their areal representation, the so-called “mapping”, for example, of evoked brain-electrical potentials.

### SUMMARY OF THE INVENTION

In the known methods and arrangements for analysis of bioelectromagnetic signals there exists the problem of so-called “biological referencing”, i.e., the correlation of the measured signals to suitable reference points for the purpose of obtaining relevant information, for example, for control of a machine, as a statement in regard to the effect of medications, or as a diagnostically relevant parameter that can be the basis for later evaluation by a physician.

Usually, for gaining relevant information based on measured signals, the signals are correlated with empirically derived data, e.g. typical averaged values, for example by examining whether a measured value is within a typical value range. However, bioelectromagnetic signals are naturally subject to a certain noise and, in regard to strength and peculiarity, differ individually so that it is often difficult to obtain meaningful information based on comparison with reference data alone.

Based on this, the invention has the object to provide a method and an arrangement for analysis of a bioelectromagnetic signal that changes over time wherein the bioelectromagnetic signal is acquired over a certain time interval and, by means of a bandpass filter, is divided into at least two signal components that differ in regard to their frequency range and that enable in a simple way to obtain information, for example, control information for controlling a machine, a prosthesis or the like, or diagnostically meaningful parameters.

With regard to the method, the object is solved by a method in which at least one reference point of a first kind is determined in accordance with predetermined selection criteria in at least one of the signal components that differ with regard to their frequency ranges and the values of at least two signal components that differ with regard to their frequency ranges at the determined reference point of the first kind are correlated with one another in accordance with predetermined evaluation criteria.

The invention is based on the surprising recognition that bioelectromagnetic signals can be used to derive important information therefrom when the bandpass filtered signals are correlated with one another in that the behavior of certain signal components is considered at characteristic reference points wherein however the reference points are not “externally” predetermined but are determined, based on predetermined selection criteria, from the detected bioelectromagnetic signal itself.

As selection criteria, the person skilled in the art advantageously has available criteria that are matched to the signal to be analyzed, for example, surpassing certain predetermined threshold values. In a preferred embodiment of the method, it is provided that the step of determining a reference point in a signal component includes the determination of possibly present extreme values and/or turning points within the time interval of the signal component. Many bioelectromagnetic signals, for example, the signal measured by means of an electroencephalograph, have characteristic curve shapes whose sections, based on turning points within a cycle, can be identified easily (for example, the so-called QRS complex or the ST segment in the electrocardiogram or the peaks generally referred to as P peaks or N peaks (P as in positive, and N as in negative) in the curve of an evoked potential in electroencephalography).

Advantageously, after the determination of at least one first reference point of the first kind in a first signal component in accordance with the same selection criteria in at least one second signal component that differs from the first one with regard to its frequency range, at least one second reference point also of the first kind is determined whereupon the values of at least two signal components that differ with regard to their frequency ranges detected at the first and second reference points of the first kind are correlated. When the signal that is being examined is, for example, the change of a potential that has been evoked as a response to a simple physical or cognitive stimulus, which change has been detected by an electroencephalograph, in this way a functional analysis of the signal can be realized, for example, in regard to the problem how individual neuronal networks whose neurons for communication with one another use different waves of a certain frequency, behave at certain points in time at which points in time networks whose neurons “send with other frequencies”, are particularly active, for example. This will be explained infra in the context of the description in connection with one embodiment.

In a further preferred embodiment, the step of correlating the values, that are assumed by different signal components at the reference point(s) of the first kind, comprises the detection of differences between the signal components and/or the detection of tendencies such as increasing or decreasing in the individual signal components. Such differences and tendencies can be visualized excellently with generally known visualization methods, optionally after solving the so-called “inverse problem” so that certain information will be visible clearly and can thus be easily read.

When the bioelectromagnetic signal to be analyzed is divided into N (NεN₊ with N₊=quantity of integers greater than 0) signal components that differ with regard to their frequency ranges, this can be done in such a way that in any of the N signal components in accordance with the same selection criteria at least one reference point of the first kind is determined. Advantageously, at least one N×N matrix of the first kind can then be generated into which the values of each signal component are entered at the N reference points of the first kind, and, based on the matrix, different functional and temporal information can be easily derived. This will also be explained in more detail in the following.

When the bioelectromagnetic signal to be analyzed has been recorded by means of a multichannel detection device in such a way that an analysis of the signal is possible with regard to spatial distribution of its sources in the examined bioelectromagnetically active object, it is advantageously possible to determine in which regions of the examined object at one of the reference points a special activity was present that resulted in a characteristic change of a certain one of the signal components that differ with regard to frequency ranges. In this connection, it can also be determined which regions of the examined object at the reference point determined with regard to a signal component are active for generating signal components of other frequencies.

In an advantageous embodiment of the method according to the invention it is provided that at least one reference point of at least one second kind, preferably of a second and a third kind, is determined. This makes it possible to select e.g. different characteristic events as the reference points. In case of encephalographic potentials that have been evoked by a visual stimulus it is possible, for example, to select as the reference points of the first kind the points in time where brain waves in the α, β, γ frequency range, that travel at different speeds within the brain, each have their own so-called C peaks; as the reference points of the second kind the points in time where the brain waves in the α, β, γ frequency range have their respective P peak; and as the reference points of the third kind the points in time where the brain waves in the α, β, γ frequency range have their N peaks.

When reference points of different kinds have been selected, the step of correlating of at least two signal components that differ in regard to their frequency ranges can advantageously also be realized for the reference points of the second kind and also the third kind, in particular, but not necessarily, based on the same evaluation criteria that can be predetermined for the reference points of the first kind.

The method has been found to be especially advantageous in regard to such bioelectromagnetic signals that are generated in reaction to an external stimulus that is supplied to the bioelectromagnetically active object that generates the bioelectromagnetic signal. This can be realized in such a way that the stimulus is supplied repeatedly to the object, that accordingly a bioelectromagnetic signal is detected repeatedly, and that the signal to be analyzed is finally derived from a suitable averaging of the detected signals. In this connection, in particular such averaging methods are suitable that do not take into account certain “outliers” in the determination of an averaged signal.

When the bioelectromagnetic signal to be analyzed is an electroencephalogram that has been recorded by means of a multichannel electroencephalograph, excellent results are obtained by using simple cognitive stimuli, in particular, simple visual stimuli, for example, a checkered pattern that changes at a certain frequency and is shown to a test person.

With regard to the arrangement, the object is solved by an arrangement for analysis of a time-variable bioelectromagnetic signal that has been detected over a certain time interval, wherein the arrangement comprises an analog or digital bandpass filter for splitting the signal into at least two signal components that differ with regard to their frequency ranges, means for automatic determination of at least one reference point of a first kind in at least one of the signal components differing in regard to the frequency ranges, and means for automatic correlation of the values of at least two signal components that differ with regard to their frequency ranges at the determined reference point of the first kind.

In an advantageous further embodiment, the means for determining a reference point can be configured such that they enable the determination of extreme values and/or turning points possibly present in an observed signal component.

Preferably, the means for determining a reference point are configured such that they enable the determination of several reference points of same or different kinds in different signal components that differ with regard to their frequency ranges.

The means for automatic correlation of the values of at least two signal components that differ with regard to their frequency ranges can be designed such that they enable the correlation of values of any signal components differing with regard to their frequency ranges at reference points of any type.

Moreover, the means for correlation of the values of different signal components at the reference point(s) can be configured such that the determination of differences between the signal components and/or the determination of tendencies such as increasing or decreasing in the individual signal components is enabled.

The bandpass filter can be configured such that it can split the bioelectromagnetic signal into N (NεN₊) signal components that differ with regard to their frequency ranges wherein N is preferably at least 3 and more preferred is 5 or 6.

When the bandpass filter enables splitting into N frequency ranges, the arrangement expediently comprises a memory unit in which at least one N×N matrix of a first kind can be written that contains the values of each signal component at N reference points of the same kind.

When the bioelectromagnetic signal has been recorded by means of a multichannel detection device such that a signal analysis with regard to spatial distribution of its sources is possible in an examined bioelectromagnetically active object, the arrangement can advantageously have means for determining and/or visualizing the regions of the examined object in which at one of the determined reference points a special activity exists that generates a certain one of the signal components that differ with regard to their frequency ranges.

The aforementioned means for determining and/or visualizing the regions of the examined object in which at one of the determined reference points a special activity exits can be designed such that a determination and/or visualization of the regions of the examined object is enabled which regions at a reference point determined for one signal component are active for generating signal components of other frequency ranges.

The further independent claims 27 to 30 each concern an advantageously embodied electroencephalograph, electromyograph, magnetoencephalograph, and electroneurograph. The independent claim 31 concerns a machine-readable memory unit containing commands required for automatically performing a method according to the invention.

Further details and advantages of the invention result from the following purely exemplary and non-limiting description of embodiments in connection with the drawing.

### BRIEF DESCRIPTION OF THE DRAWINGS

FIG. 1 shows a schematic that illustrates the prior art (a) based on an example of an electroencephalographically measured visually evoked potential in comparison to the basic principle of the invention (b).

FIG. 2 is a schematic diagrammatic illustration showing the classic course of the electroencephalographic measurement of a visually evoked potential.

FIG. 3 shows in four parts a) to d) purely schematically the basic method steps for obtaining the inventively treated electroencephalographic data of visually evoked potentials.

FIG. 4 shows purely schematically the selection of reference points of first, second, and third kind and of the correlation of the values of the different signal components that differ with regard to their frequency ranges relative to the reference points.

FIG. 5 shows purely schematically the configuration of a N×N matrix according to an advantageous embodiment of the invention.

FIG. 6 shows purely schematically some of the information that is obtainable after solving the inverse problem based on the example of electroencephalographic data, in accordance with the prior art (FIG. 6a) and in accordance with the method of the present invention (FIG. 6b).

FIG. 7 shows, based on the example of electroencephalographic data in the form of section images, the information (7b) obtainable by the present invention in comparison to the prior art (FIG. 7a).

### DESCRIPTION OF PREFERRED EMBODIMENTS

In the following the invention will be explained based on an example applied to electroencephalographically measured data.

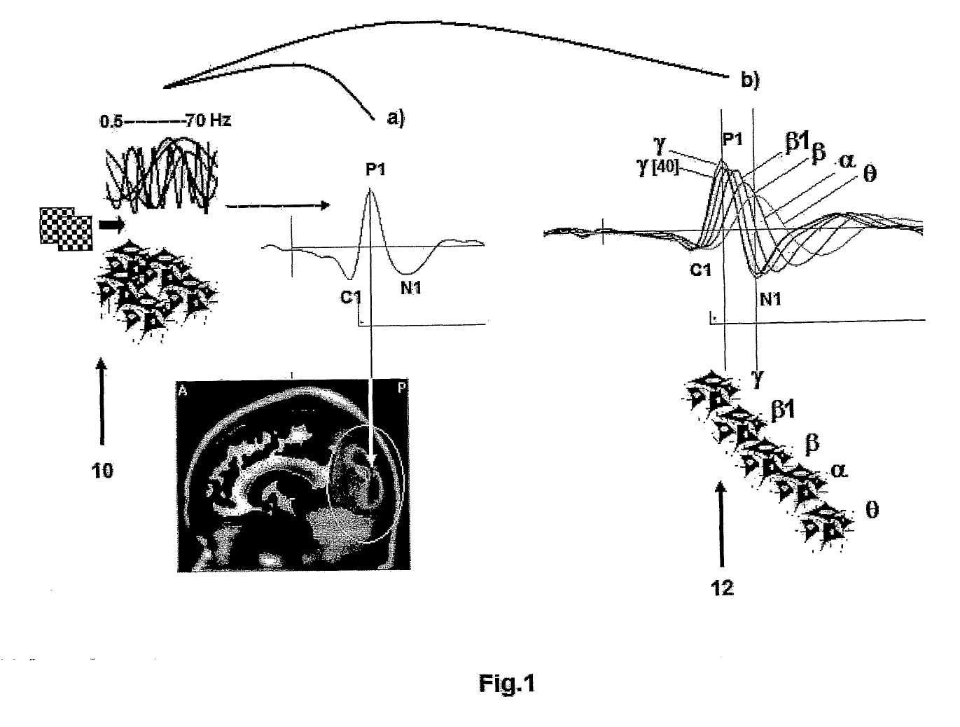

FIG. 1 shows schematically the course of obtaining such electroencephalographic data by visually evoked potentials in the brain. For this purpose, a test person is shown over a certain period of time a checkered pattern in which at a certain frequency, typically 1 Hz, the fields change their color, i.e., black fields turn white and white fields turn black.

This simple visual stimulus evokes in the brain of the test person a potential and thus a bioelectric signal within the meaning in accordance with the present invention; the signal can be measured by a conventional electroencephalograph, for which purpose e.g. 30Ag/AgCl electrodes are positioned by employing the international 10/10 electrodes placement system on different points of the skull of the test person.

As already mentioned, the neurons in the brain form different neuronal networks (indicated by reference numeral 10 in FIG. 1) wherein they use waves of different frequencies for communication with one another; the frequencies are essentially within a range between 0.5 and 70 Hz. As already mentioned, this frequency range is usually divided into certain sub-ranges so that the individual networks can be characterized according to the frequencies of the waves that are used by their neurons for communicating with one another as γ, α, β, β1 and Θ networks. In the signal measured by the electrodes the different components can be isolated by means of the bandpass filter.

In the classic treatment, these different networks are not taken into account in the evaluation of the measured data: as indicated in FIG. 1 at a), averaging across the entire measured values is used for forming a single graph in which the course of the measured potential over time is plotted and in which the prominent turning points that are usually referred to as P peaks and N peaks are sequentially numbered beginning at 1 (P1, N1, etc.). The course of the graph is in general approximately such that approximately 70 milliseconds after stimulation a first distinctive extreme value in the negative potential range occurs (identified in FIG. 1 at “C1”), approximately 100 milliseconds after the stimulation the first peak in the positive range (P1) occurs, and approximately 140 milliseconds after the stimulation the so-called N1 peak occurs within the negative potential range.

After solving the so-called “inverse problem”, i.e., the calculation based on the data measured at the surface of the skull where in the brain a potential has been evoked, an image can be displayed as shown in FIG. 1, for example, in the form of a section image or a virtual three-dimensional image of the skull and the brain, wherein the area in which on average the strongest activity was observed is graphically enhanced in the image.

Surprisingly it has now been found that important information can be gained when the aforementioned networks are considered separately and their behavior as a response to e.g. the aforementioned visual stimulus are correlated with one another at characteristic reference points. As illustrated in FIG. 1 at b), the individual components in the signal measured by the electrodes are considered by splitting the signal into the different frequency ranges so that, for example, five or six graphs are obtained or one graph with five or six potential courses that, as indicated in FIG. 1 at b), are identified by γ, γ[40], α, β, β1, and Θ and that, as also shown in FIG. 1 at b) enable identification with regard to the networks based on the different frequencies used by the neurons (indicated with reference numeral 12 in FIG. 1).

Since the differences between the γ, γ[40] networks are only small, the network γ[40] is not shown in the schematic.

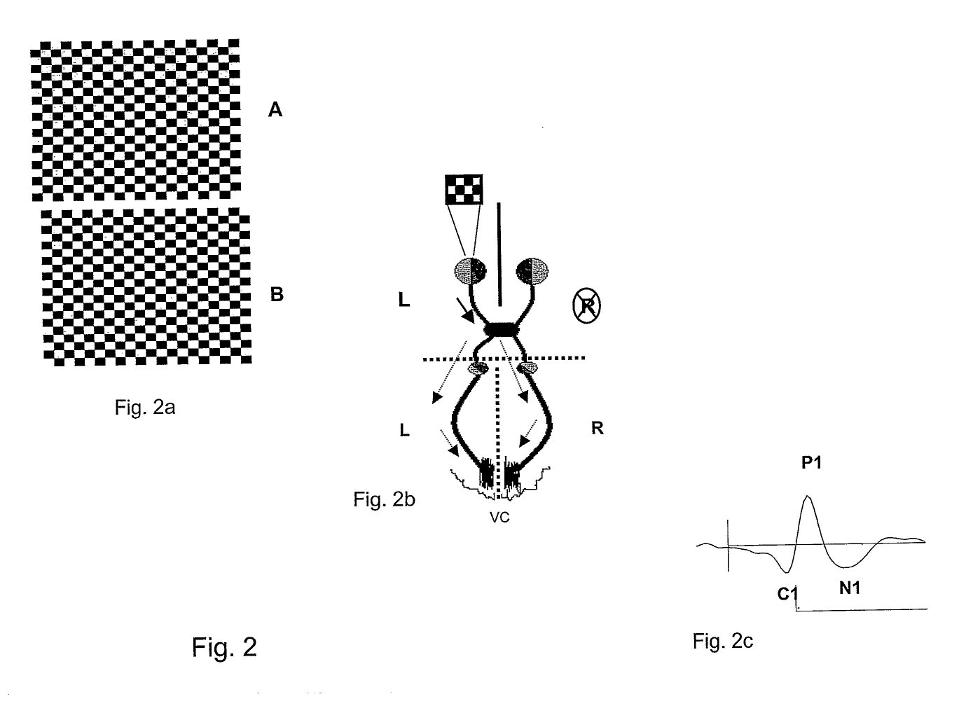

In FIG. 2, the classic course of the electroencephalographic measurement of a visually evoked potential is illustrated. FIG. 2a shows the two different checkered patterns A and B that, as indicated in FIG. 2b, alternatingly, for example at a frequency of 1 Hz, are shown to the eye of a test person, in the example of FIG. 2b to the left eye.

By means of this stimulus, an electrical activity is generated in the so-called visual cortex (in FIG. 2b identified at “VC”) of the brain of the test person. Usually, this electrical activity is converted into a graph as shown in FIG. 2c after averaging over a certain time interval, for example, 60 seconds.

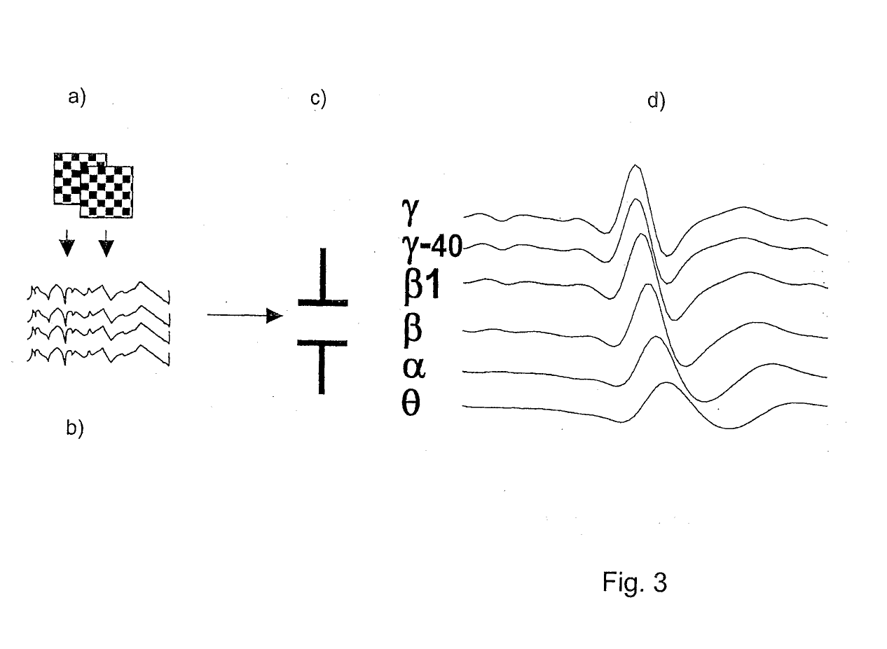

FIG. 3 shows in four parts purely schematically the basic method steps for obtaining the electroencephalographic data analyzed in accordance with the invention.

The already described checkered pattern is shown to a person, not illustrated in detail in this context (FIG. 3a).

This visual stimulus generates in the brain of the test person an electrical activity, i.e., the neurons of different neuronal networks are activated wherein, for communication with one another, they use waves of different frequencies (schematically indicated in FIG. 3b).

This activity that can be measured in the manner known in the art by conventional electroencephalographs on the exterior of the skull is now filtered by bandpass filter (FIG. 3c) so that the electroencephalographic signal with regard to different frequency ranges, as indicated in FIG. 3d, can be split into different signal components.

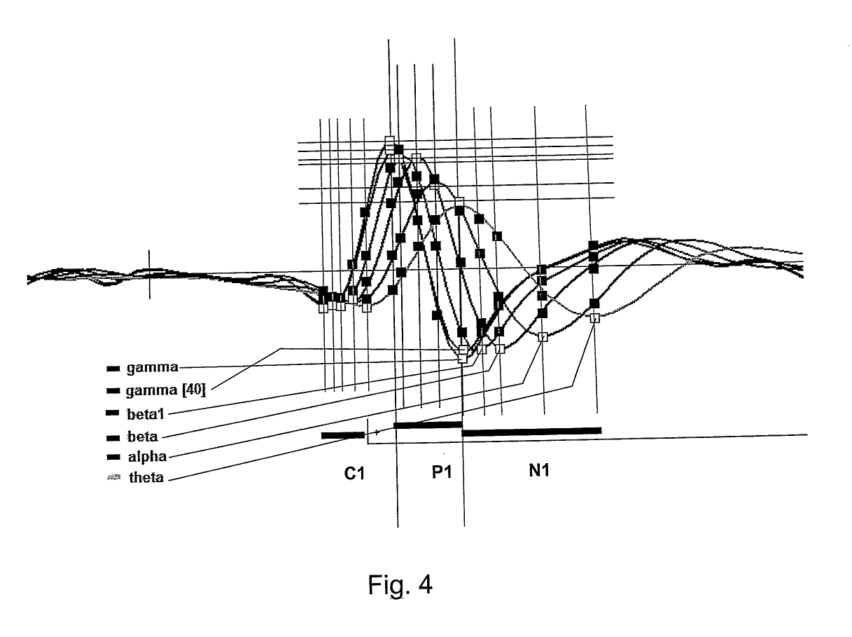

By splitting the measured signals into the different signal components it is possible to identify different networks in the brain. Surprisingly, it has been found that important information, for example, for controlling a machine, a prosthesis, and the like, in particular also diagnostically meaningful parameters, can be obtained when the different signal components are correlated with one another and not, as has been done in the past, to any kind of a comparative group, as indicated in FIG. 4.

For this purpose, in the individual signal components γ, γ[40], α, β, β1, and Θ first certain reference points, in the illustrated embodiments reference points of the first, second, and third kind, are selected, wherein in particular as reference points of the first kind, respectively, the points in time at which the signal components have reached the C1 peaks; as reference points of the second kind, respectively, those points in time at which the signal components have reached the P1 peaks; and as reference points of the third kind, respectively, those points in time at which the signal components have reached the N1 peaks are selected.

Is should be noted here that, of course, depending on the type of stimulus (visual, acoustic, olfactory, gustatory, tactile etc.) and the type of measurement (electroencephalographic, electrocardiographic etc.) and depending on the problem, entirely different reference points can be selected. Important is that in the different signal components reference points are selected at all, relative to which the behavior of the other signal components is examined, i.e., the values of the signal components that differ with regard to the frequency ranges are correlated with one another at the selected reference points in accordance with predetermined selection criteria. Such selection criteria can be the determination of differences between the signal components and/or determination of tendency such as increasing or decreasing within the individual signal components.

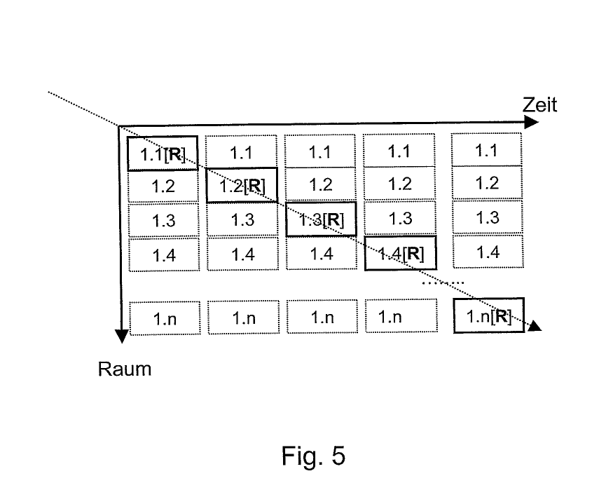

Subsequently, as shown in FIG. 5, an N×N matrix is generated in which the diagonal represents the reference points of the same kind, i.e., for example, the P1 peaks. On the diagonal of the matrix the values of those signal components are listed that at the respective point in time just reach their own P1 peaks. In the rows of the matrix the values of one signal component are entered which values the signal component has at the points in time at which they themselves or the other signal components have reached e.g. the P1 peak. In the columns of the matrix the values of the different signal components are entered which values result at the point in time at which one of the signal components reaches, for example, the P1 peak. In this way, a spatial-temporal resolution results wherein a special feature is that the reference points are dynamic, i.e., they are not set at predetermined time intervals but are dynamically selected always when a signal component passes through its own P1 peak. Moreover, since the other signal components are then correlated with this signal component, and not to external comparative data, this can be referred to as “dynamic self referencing”.

In the matrix two different kinds of information are thus contained: one describes the temporal development of each network, the other indicates the contextual interaction of the networks at certain points in time. Both data sets have different functional implications that can be demonstrated by statistic dependencies.

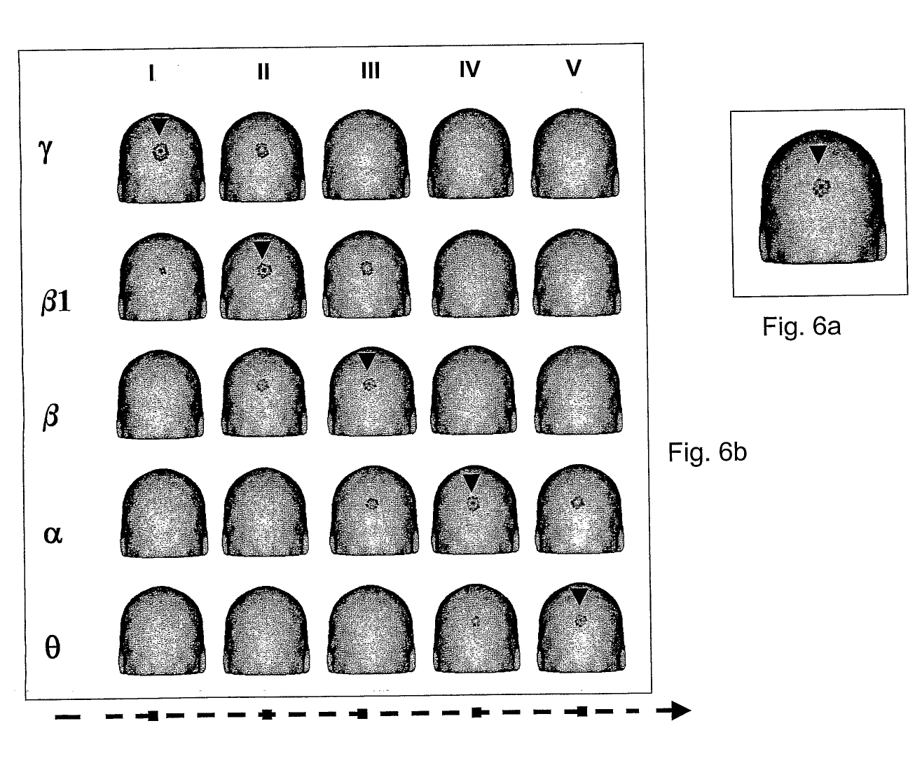

By solving the aforementioned inverse problem and corresponding visualization techniques, virtual 3-D images, shown in FIG. 6 naturally only two-dimensionally, can be generated that contain important information. In the classic treatment, after averaging all signal components only one image such as shown in FIG. 6a would be obtained in which the average behavior of all different networks at a certain reference point, for example the P1 peak, is illustrated.

When however the signal components, for example, are split into five different frequency ranges and the signal components are analyzed as described above at the selected reference points, 25 images are obtained, as shown in FIG. 6b, that visualize the behavior of the different networks at different reference points. In FIG. 6b the black triangle indicates the network that provides momentarily a reference point, i.e., the P1 peak. Horizontally adjacent the temporal development of the same networks can be read at the points in time at which the other networks pass through the respective P1 peak.

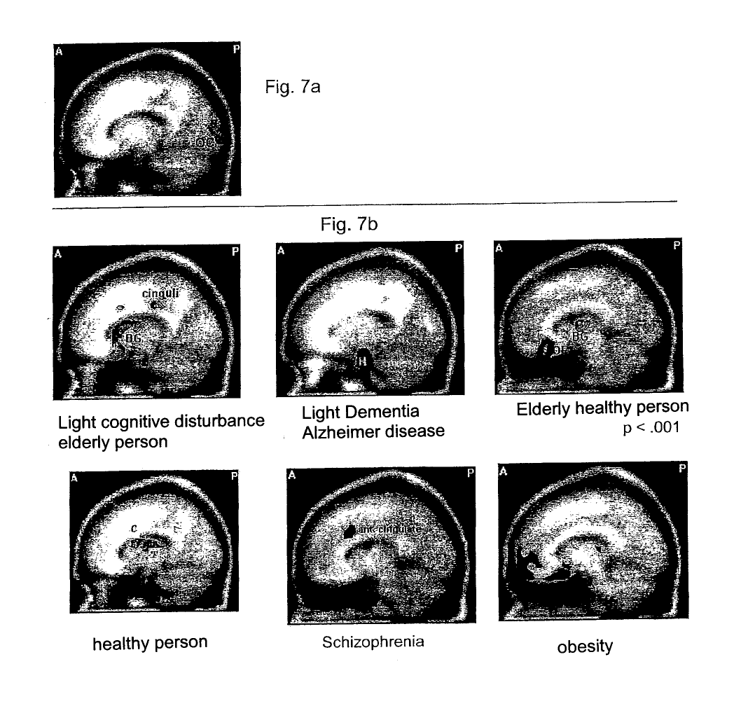

Surprisingly it has been found that certain pathologic or other changes effected by medication in the behavior of the networks cannot be determined by classic treatment because after averaging and correlation to external reference data no deviations are detectable; but by means of dynamic self referencing certain disease patterns or certain medications exhibit significant differences in the spatial/temporal development of the different network activities so that, for example, in persons suffering from a certain disease or predisposed to such a disease at a point in time when a certain network passes through the P1 peak already a different network is active in a certain area while such an activity in healthy persons will not be exhibited. This is impressively shown in FIGS. 7a and 7b: while ill and healthy persons in an evaluation of electroencephalographically obtained data in accordance with the prior art show no significant deviations (FIG. 7a), the inventive evaluation of the same data shows in patients with different diseases significantly different activities in different areas (FIG. 7b).

It has also been found that not only the spatial but also the temporal behavior of different networks can contain revealing information that by means of the invention can be obtained for the first time and can be made available for further evaluation, for example, by a physician. It has been found that under certain testing conditions between healthy and ill patients no deviations with regard to spatial activation of certain areas in the brain can be observed but a difference in the temporal behavior of the networks at the dynamic reference points can be observed.

The invention enables thus advantageously also screening tests and early detection and can also be used with advantage for developing and in particular testing new medications.

It should be noted that the invention also implies new business methods, in particular the sale of analyses of bioelectromagnetic, in particular electroencephalographic signals, wherein these methods are expressly denoted as belonging to the invention and are claimed in those countries where permitted by national laws.

What is claimed is:

1.-32. (canceled)

33. Method for analysis of a time-variable bioelectromagnetic signal that has been recorded over a certain time interval, comprising the steps of:
splitting the signal by a bandpass filter into at least two signal components that differ with regard to their frequency ranges;
determining in at least one of the signal components that differ with regard to their frequency ranges at least one reference point of a first kind in accordance with predetermined selection criteria; and
correlating the values of at least two signal components that differ with regard to their frequency ranges at the determined reference points of the first kind with one another according to predetermined evaluation criteria.

34. Method according to claim 33, wherein the step of determining a reference point in a signal component comprises the determination of extreme values and/or turning points in the signal component that are possibly present in the time interval.

35. Method according to claim 33, wherein at least one first reference point of the first kind is determined in a first signal component; according to the same selection criteria in at least one second signal component that differs from the first one with regard to the frequency range at least one second reference point of the first kind is determined; and the values of at least two signal components that differ with regard to their frequency ranges in the first and second reference points of the first kind are correlated with one another.

36. Method according to claim 35, wherein the step of correlating of the values that are derived from different signal components at the reference point or points of the first kind encompasses the determination of differences between the signal components and/or the determination of tendencies such as increasing or decreasing in the individual signal components.

37. Method according to claim 33, wherein N (NεN₊) signal components that differ with regard to their frequency ranges of a bioelectromagnetic signal are analyzed, wherein in each of the N signal components according to the same selection criteria at least one reference point of the first kind is determined.

38. Method according to claim 37, further comprising the step of generating at least one N×N matrix of a first kind into which matrix the values of each signal component is entered at the N reference points of the first kind.

39. Method according to claim 33, wherein the bioelectromagnetic signal has been recorded by means of a multichannel detection device such that an analysis of the signal with regard to spatial distribution of its sources in an examined bioelectromagnetic active object is possible, wherein a determination is made in which regions of the examined object at one of the determined reference points a special signal activity resulting in the generation of a certain one of the signal components that differ with regard to their frequency.

40. Method according to claim 39, wherein it is determined which regions of the examined objects are active at the reference point determined with regard to a signal component for generating signal components of other frequency ranges.

41. Method according to claim 40, wherein at least one reference point of at least one second kind, preferred a second and a third kind, are determined.

42. Method according to claim 41, wherein the steps of correlating at least two signal components that differ with regard to their frequency ranges are also carried out for the reference points of the second and optionally third kind in accordance with evaluation criteria that are not necessarily predetermined identically.

43. Method according to claim 33, wherein the bioelectromagnetic signal to be analyzed is a signal that is generated as a response to a stimulus externally applied to the bioelectromagnetically active object that produces the bioelectromagnetic signal.

44. Method according to claim 43, wherein the bioelectromagnetic signal to be examined is a signal resulting from averaging the signals that are recorded, respectively, after multiple application of the stimulus.

45. Method according to claim 33, wherein the bioelectromagnetic signal is an electroencephalogram that is recorded with a multichannel electroencephalograph.

46. Method according to claim 45, wherein the stimulus is a physical/cognitive stimulus selected from the group consisting of a visual, a tactile, a gustatory, an olfactory, and an acoustic stimulus.

47. Method according to claim 46, wherein the stimulus is a checkered pattern changing in accordance with a certain frequency.

48. Method according to claim 45, wherein the signal is a signal resulting from a reaction to a physical/cognitive stimulus, wherein the extreme values that are generally referred to as P and N peaks, wherein as a reference point of the first kind the points in time are selected at which the signal components reached the P peaks, and as a reference point of the second kind the points in time are selected at which the signal components reach the N peaks.

49. Method according to claim 33, wherein the signal to be analyzed is split by means of bandpass filtering into at least three, preferably into five to six, frequency ranges.

50. Arrangement for analysis of a time-variable bioelectromagnetic signal that is recorded over a certain time interval, the arrangement comprising:
an analog or digital bandpass filter for splitting the signal into at least two signal components that differ with regard to their frequency ranges;
means for automatically determining at least one reference point of a first kind in at least one of the signal components that differ with regard to their frequency ranges; and
means for automatically correlating values of at least two of the signal components that differ with regard to their frequency ranges at the certain reference point of the first kind are provided.

51. Arrangement according to claim 50, wherein the means for automatically determining are designed such that said means enable determination of extreme values and/or turning points possibly present in the signal component that is being observed.

52. Arrangement according to claim 50, wherein the means for automatically determining are designed such that said means enable determination of several reference points of the same or different kind indifferent signal components that differ with regard to their frequency ranges.

53. Arrangement according to claim 50, wherein the means for automatically correlating are designed such that they enable correlation of values of any signal components that differ with regard to their frequency ranges at reference points of any kind.

54. Arrangement according to claim 50, wherein the means for automatically correlating are designed such that they enable detection of differences between the signal components and/or the determination of tendencies such as increasing or decreasing in the individual signal components.

55. Arrangement according to claim 50, wherein the bandpass filter is designed to split the bioelectromagnetic signal into N (NεN₊) signal components that differ with regard to their frequency ranges therein N is preferably at least equal to 3, and more preferred equal to 5 or 6.

56. Arrangement according to claim 55, wherein a memory unit is provided into which at least one N×N matrix of the first kind can be written that contains the values of each signal component at N reference points of the same kind.

57. Arrangement according to claim 50, wherein the bioelectromagnetic signal is recorded by means of a multichannel detection device in such a way that an analysis of the signals with regard to spatial distribution of its sources in an examined bioelectromagnetically active object is enabled, the arrangement further comprising means for determination and/or visualization of the regions of the examined object in which at a certain reference point a special activity is present that results in generation of a certain one of the signal components that differ with regard to their frequency ranges.

58. Arrangement according to claim 50, wherein the means for determination and/or visualization of the regions of the examined object in which at a certain one of the reference points a special activity is present are designed such that a determination and/or visualization of the regions of the examined objects is enabled that are active at a reference point that has been determined relative to one signal component for generating signal component of other frequency ranges.

59. Arrangement according to claim 50, in the form of an electroencephalograph, an electromyograph, an electroneurograph, or a magnetoencephalograph.
